(19) United States
(12) Patent Application Publication
(10) Pub. No.: US 2023/0000150 A1
(43) Pub. Date: Jan. 5, 2023

(54) DROPLET DELIVERY DEVICE WITH OPTIMIZED MIXING OF SUSPENSIONS

(71) Applicant: PNEUMA RESPIRATORY, INC., Boone, NC (US)

(72) Inventors: John H. Hebrank, Boone, NC (US); Judson Sidney Clements, Boone, NC (US)

(73) Assignee: PNEUMA RESPIRATORY, INC., Boone, NC (US)

(21) Appl. No.: 17/852,742

(22) Filed: Jun. 29, 2022

Related U.S. Application Data
(60) Provisional application No. 63/216,651, filed on Jun. 30, 2021.

(51) Int. Cl. A24F 40/05; A24F 40/48; A24F 40/42; A24F 40/60

(52) U.S. Cl. CPC A24F 40/05; A24F 40/48; A24F 40/42; A24F 40/60

(57) ABSTRACT

A droplet delivery device with an ejector mechanism and fluid reservoir includes an accelerometer to determine and communicate optimized mixing of fluid in the reservoir for inhalation of droplets, such as into the lungs, from the droplet delivery device.

### CROSS-REFERENCE TO RELATED APPLICATIONS

The present application claims the benefit of priority of U.S. Provisional Application No. 63/216,651 filed Jun. 30, 2021, which is incorporated by reference in its entirety.

### FIELD OF THE INVENTION

This disclosure relates to droplet delivery devices and more specifically to droplet delivery devices for the delivery of fluids to the pulmonary system.

### BACKGROUND OF THE INVENTION

The use of aerosol generating devices for the treatment of a variety of respiratory diseases is an area of large interest. Inhalation provides for the delivery of aerosolized drugs to treat asthma, COPD and site-specific conditions, with reduced systemic adverse effects. A major challenge is providing a device that delivers an accurate, consistent, and verifiable dose, with a droplet size that is suitable for successful delivery of medication to the targeted lung passageways.

Dose verification, delivery, and inhalation of the correct dose at prescribed times is important. Getting patients to use inhalers correctly is also a major problem. A need exists to ensure that patients correctly use inhalers and that they administer the proper dose at prescribed times. Problems emerge when patients misuse or incorrectly administer a dose of their medication. Unexpected consequences occur when the patient stops taking medications, owing to not feeling any benefit, or when not seeing expected benefits or overuse the medication and increase the risk of over dosage. Physicians also face the problem of how to interpret and diagnose the prescribed treatment when the therapeutic result is not obtained.

Currently most inhaler systems such as metered dose inhalers (MDI) and pressurized metered dose inhalers (p-MDI) or pneumatic and ultrasonic-driven devices generally produce drops with high velocities and a wide range of droplet sizes including large droplet that have high momentum and kinetic energy. Droplets and aerosols with such high momentum do not reach the distal lung or lower pulmonary passageways but are deposited in the mouth and throat. As a result, larger total drug doses are required to achieve the desired deposition in targeted areas. These large doses increase the probability of unwanted side effects.

Aerosol plumes generated from current aerosol delivery systems, as a result of their high ejection velocities and the rapid expansion of the drug carrying propellant, may lead to localized cooling and subsequent condensation, deposition and crystallization of drug onto the ejector surfaces. Blockage of ejector apertures by deposited drug residue is also problematic.

This phenomenon of surface condensation is also a challenge for existing vibrating mesh or aperture plate nebulizers that are available on the market. In these systems, in order to prevent a buildup of drug onto mesh aperture surfaces, manufacturers require repeated washing and cleaning, as well as disinfection after a single use in order to prevent possible microbiological contamination. Other challenges include delivery of viscous drugs and suspensions that can clog the apertures or pores and lead to inefficiency or inaccurate drug delivery to patients or render the device inoperable. Also, the use of detergents or other cleaning or sterilizing fluids may damage the ejector mechanism or other parts of the nebulizer and lead to uncertainty as to the ability of the device to deliver a correct dose to the patient or state of performance of the device.

Accordingly, there is a need for an inhaler device that delivers droplets of a suitable size range, avoids surface fluid deposition and blockage of apertures, with a dose that is verifiable, and provides feedback regarding correct and consistent usage of the inhaler to patient and professional such as physician, pharmacist or therapist.

### SUMMARY OF THE INVENTION

In an embodiment of the invention, a droplet delivery device comprised a housing with an outlet configured for droplets to be ejected from the droplet delivery device; a reservoir configured to supply a volume of fluid and in fluid communication with the outlet; an ejector mechanism in fluid communication with the reservoir and the outlet; an accelerometer coupled to a power source and to the housing; a microcontroller unit coupled to the accelerometer and programmed to communicate a confirmation of sufficient movement of the droplet delivery device to mix the volume of fluid; and a feedback unit communicatively coupled to the microcontroller unit providing one or both of display and sound notification in response to receiving the confirmation of sufficient movement.

In another embodiment of the invention, an accelerometer of a droplet delivery device is configured to measure at least one of acceleration and orientation on multiple axes.

In another embodiment of the invention, an accelerometer of a droplet delivery device is configured to measure at least one of acceleration and orientation on at least three orthogonal axes.

In another embodiment of the invention, a droplet delivery device with an accelerometer has an ejector mechanism that includes an electromechanical actuator.

In another embodiment of the invention, a droplet delivery device with an accelerometer has a reservoir containing a fluid including a drug.

In another embodiment of the invention, a droplet delivery device with an accelerometer has a reservoir containing a fluid including nicotine or a cannabinoid.

In another embodiment of the invention, a droplet delivery device with an accelerometer has a reservoir containing a fluid including a therapeutic agent.

In an embodiment of the invention, a droplet delivery device with an accelerometer includes a feedback unit directly or indirectly coupled to the accelerometer that is configured to provide a voice notification.

In an embodiment of the invention, a droplet delivery device with an accelerometer includes a microcontroller unit that is programmed to initiate the accelerometer to determine at least one of acceleration and orientation at predetermined intervals. In further embodiments, the microcontroller unit is programmed to wake the accelerometer to initiate the accelerometer to determine at least one of acceleration and orientation at predetermined intervals.

In an embodiment of the invention, a droplet delivery device with an accelerometer includes a microcontroller unit that is programmed to communicate directions for moving the droplet delivery device to a feedback unit based on one or more determinations of at least one of acceleration and orientation at one or more predetermined intervals.

In an embodiment of the invention, a droplet delivery device with an accelerometer includes a microcontroller unit that is programmed to communicate directions for moving the droplet delivery device to a feedback unit based on one or more determinations of at least one of acceleration and orientation at one or more predetermined intervals.

In an embodiment of the invention, a droplet delivery device with an accelerometer includes a microcontroller unit coupled to a recordable memory, wherein the microcontroller unit is programmed to store orientation data from the accelerometer relative to time in the recordable memory.

In an embodiment of the invention, a droplet delivery device with an accelerometer includes a microcontroller unit coupled to a recordable memory, wherein the microcontroller unit is programmed to store movement data from the accelerometer to the recordable memory.

In embodiments of the invention, a droplet delivery device with an accelerometer includes an ejector mechanism configured to produce droplets with an average ejected droplet diameter of less than about 6 microns.

In embodiments of the invention, a droplet delivery device with an accelerometer includes an ejector mechanism configured to produce droplets with an average ejected droplet diameter of less than about 5 microns.

In embodiments of the invention, a droplet delivery device with an accelerometer includes an ejector mechanism configured to produce droplets with an average ejected droplet diameter of more than about 1 micron to less than about 6 microns.

In embodiments of the invention, a droplet delivery device with an accelerometer includes an ejector mechanism configured to produce droplets with an average ejected droplet diameter of more than about 1 micron to less than about 5 microns.

### DETAILED DESCRIPTION OF THE INVENTION

For MDI's and nebulizers the active drug component frequently has a density different from the fluid carrier. The active drug may be in solution or in suspension. If it is in suspension, the density difference will cause the drug to “settle out” so that when dispensed, the active drug level may be greater than or less than the average dose depending upon where the active drug has settled.

Standard practice with inhalers using suspensions of drugs is for the patient to vigorously shake the device containing the drug for 15 times immediately prior to use. However, many patients neglect or misunderstand the correct procedure resulting in drug dosages being too high or too low. There are different effects on drug dose when the drug is shaked correctly, or weakly or not at all, as well as how much dose error occurs if the patient waits to dispense after shaking. For example, weakly shaking an MDI may result in up to an 80% overdose and single inversion before use may result in a 40% overdose of crystalline drug dispensed from an MDI. Similarly, use an hour after shaking can result in a 20% overdose. Co-suspension technology has somewhat mitigated this problem, reducing the respective overdoses to 20%, 20% and 10% by reducing the difference in density, hence settling, between the drug crystals and the drug carrier fluid.

Embodiments of the invention remedy dose errors caused by incorrect use of the inhaler by monitoring correct shaking and informing the patient whether shaking, i.e. movement of the droplet delivery device and housed fluid reservoir, has been sufficient and recently enough for good mixing of the drug. An accelerometer is mounted in the MDI or droplet delivery device which can measure the intensity of shaking to confirm that there have been enough shakes and shakes were vigorous enough. Typically, “vigorous” means that accelerations of the shaking were of sufficient magnitude to stir the fluid and crystals into a uniform suspension. In normal use, the patient turns on the device, activating the acceleration measurement, then shakes the device and after confirmation of a correct shaking, either by display or sound (including voice) from a visual or audio feedback unit (including a mobile device such as a smartphone connected via wired or wireless connection to a microcontroller unit coupled to the accelerometer), dispenses the drug dose during an inhalation. Typically, a good shake is determined, which may vary depending type of drug or suspension being used, confirming that there are sufficient shakes of sufficient magnitude. If shaking is not sufficient, according to the algorithm, the device can request the patient to shake the device again, or more times, or more vigorously. The device can also archive whether the patient has shaken the device properly at the time of each dosage to alert their medical provider that shaking is not being done.

Where shaking needs to be along the long axis of the device, this can be monitored by the accelerometer. Typically, accelerometers measure acceleration in multiple axes, preferably three orthogonal axes (X, Y and Z) so if there is a preferred mix of shaking, it can be measured and confirmed.

Where shaking is more effective when the orientation of recent settling is known, the accelerometer can regularly measure the orientation of the device. For example, the device can “wake up” every ten minutes and record the acceleration and/or orientation of the device in all three axes. An algorithm can then determine the orientation with respect to gravity. Orientation data can be archived so it is known that device has been in a single orientation for a long period of time and therefore needs to be shaken in a particular direction with more or less vigor, or more, shakes. In such embodiments, the device includes electronics that “wake up” to make the measurement, a two or three axis accelerometer, memory to store results, a method and notification software/hardware to inform the patient of the results, and optionally an internal clock to time stamp each reading.

To implement the above functions, a STMicroelectronics (Geneva, Switzerland) LIS3DH three-axis accelerometer may be used to detect device orientation and shaking. The LIS3DH reads acceleration to a resolution of 30 milliG in the 10-bit mode with a +−16 G full scale. The device MCU microcontroller is capable of reading the accelerometer data at a rate sufficient for a 600 Hz bandwidth, however rates of 300 Hz are sufficient for active measurement of the device being shaken. In normal operation lower data rates are initially used to the accelerometer until an active shake is detected (acceleration larger than 0.1 G) and then the data rate is increased for accurate assessment of the shaking. An I2C connection is used to communicate between the accelerometer and microcontroller unit (MCU). In embodiments, the board containing the MCU and accelerometer also include a clock and a sound chip with speaker, and a Bluetooth chip capable of delivering compliance data to a smartphone. The board is battery powered and “wakes up” once every ten minutes to monitor device position. The preferred device in embodiments can monitor longer term orientation of the device as well as measuring, recording and communicating the magnitude, movement data as to acceleration and orientation, including number of shakes and time of shaking, as well as the length of time prior to use that the device was oriented in substantially the same position.

Effective delivery of medication to the deep pulmonary regions of the lungs through the alveoli, has always posed a problem, especially to children and elderly, as well as to those with the diseased state, owing to their limited lung capacity and constriction of the breathing passageways. The impact of constricted lung passageways limits deep inspiration and synchronization of the administered dose with the inspiration/expiration cycle. For optimum deposition in alveolar airways, droplets with aerodynamic diameters in the ranges of 1 to 5 μm are optimal, with droplets below about 4 μm shown to reach the alveolar region of the lungs, while larger droplets are deposited on the tongue or strike the throat and coat the bronchial passages. Smaller droplets, for example less than about 1 μm, can penetrate more deeply into the lungs but have a tendency to be exhaled.

In certain aspects, the present disclosure relates to an in-line droplet delivery device for delivery of a fluid as an ejected stream of droplets to the pulmonary system of a subject and related methods of delivering safe, suitable, and repeatable dosages to the pulmonary system of a subject. The present disclosure also includes an in-line droplet delivery device and system capable of delivering a defined volume of fluid in the form of an ejected stream of droplets such that an adequate and repeatable high percentage of the droplets are delivered into the desired location within the airways, e.g., the alveolar airways of the subject during use.

The present disclosure provides an in-line droplet delivery device for delivery of a fluid as an ejected stream of droplets to the pulmonary system of a subject, the device comprising a housing, a reservoir for receiving a volume of fluid, and an ejector mechanism including a piezoelectric actuator and an aperture plate, wherein the ejector mechanism is configured to eject a stream of droplets having an average ejected droplet diameter of less than about 5-6 microns, preferably less than about 5 microns. As shown in further detail herein, the droplet delivery device is configured in an in-line orientation in that the housing, ejector mechanism and related electronic components are orientated in a generally in-line or parallel configuration so as to form a small, hand-held device.

In specific embodiments, the ejector mechanism is electronically breath-activated by at least one differential pressure sensor located within the housing of the in-line droplet delivery device upon sensing a pre-determined pressure change within the housing. In certain embodiments, such a pre-determined pressure change may be sensed during an inspiration cycle by a user of the device, as will be explained in further detail herein.

In accordance with certain aspects of the disclosure, effective deposition into the lungs generally requires droplets less than about 5-6 μm in diameter. Without intending to be limited by theory, to deliver fluid to the lungs, a droplet delivery device must impart a momentum that is sufficiently high to permit ejection out of the device, but sufficiently low to prevent deposition on the tongue or in the back of the throat. Droplets below approximately 5-6 μm in diameter are transported almost completely by motion of the airstream and entrained air that carry them and not by their own momentum.

In certain aspects, the present disclosure includes and provides an ejector mechanism configured to eject a stream of droplets within the respirable range of less than about 5-6 μm, preferably less than about 5 μm. The ejector mechanism is comprised of an aperture plate that is directly or indirectly coupled to a piezoelectric actuator. In certain implementations, the aperture plate may be coupled to an actuator plate that is coupled to the piezoelectric actuator. The aperture plate generally includes a plurality of openings formed through its thickness and the piezoelectric actuator directly or indirectly (e.g. via an actuator plate) oscillates the aperture plate, having fluid in contact with one surface of the aperture plate, at a frequency and voltage to generate a directed aerosol stream of droplets through the openings of the aperture plate into the lungs, as the patient inhales. In other implementations where the aperture plate is coupled to the actuator plate, the actuator plate is oscillated by the piezoelectric oscillator at a frequency and voltage to generate a directed aerosol stream or plume of aerosol droplets.

In certain aspects, the present disclosure relates to an in-line droplet delivery device for delivering a fluid as an ejected stream of droplets to the pulmonary system of a subject. In certain aspects, the therapeutic agents may be delivered at a high dose concentration and efficacy, as compared to alternative dosing routes and standard inhalation technologies.

In certain embodiments, the in-line droplet delivery devices of the disclosure may be used to treat various diseases, disorders and conditions by delivering therapeutic agents to the pulmonary system of a subject. In this regard, the in-line droplet delivery devices may be used to deliver therapeutic agents both locally to the pulmonary system, and systemically to the body.

More specifically, the in-line droplet delivery device may be used to deliver therapeutic agents as an ejected stream of droplets to the pulmonary system of a subject for the treatment or prevention of pulmonary diseases or disorders such as asthma, chronic obstructive pulmonary diseases (COPD) cystic fibrosis (CF), tuberculosis, chronic bronchitis, or pneumonia. In certain embodiments, the in-line droplet delivery device may be used to deliver therapeutic agents such as COPD medications, asthma medications, or antibiotics. By way of non-limiting example, such therapeutic agents include albuterol sulfate, ipratropium bromide, tobramycin, and combinations thereof.

In other embodiments, the in-line droplet delivery device may be used for the systemic delivery of therapeutic agents including small molecules, therapeutic peptides, proteins, antibodies, and other bioengineered molecules via the pulmonary system. By way of non-limiting example, the in-line droplet delivery device may be used to systemically deliver therapeutic agents for the treatment or prevention of indications inducing, e.g., diabetes mellitus, rheumatoid arthritis, plaque psoriasis, Crohn's disease, hormone replacement, neutropenia, nausea, influenza, etc.

By way of non-limiting example, therapeutic peptides, proteins, antibodies, and other bioengineered molecules include: growth factors, insulin, vaccines (Prevnor—Pneumonia, Gardasil—HPV), antibodies (Avastin, Humira, Remicade, Herceptin), Fc Fusion Proteins (Enbrel, Orencia), hormones (Elonva—long acting FSH, Growth Hormone), enzymes (Pulmozyme—rHu-DNAase-), other proteins (Clotting factors, Interleukins, Albumin), gene therapy and RNAi, cell therapy (Provenge—Prostate cancer vaccine), antibody drug conjugates—Adcetris (Brentuximab vedotin for HL), cytokines, anti-infective agents, polynucleotides, oligonucleotides (e.g., gene vectors), or any combination thereof; or solid droplets or suspensions such as Flonase (fluticasone propionate) or Advair (fluticasone propionate and salmeterol xinafoate).

In other embodiments, the in-line droplet delivery device of the disclosure may be used to deliver a solution of nicotine including the water-nicotine azeotrope for the delivery of highly controlled dosages for smoking cessation or a condition requiring medical or veterinary treatment. In addition, the fluid may contain cannabinoids (such as THC, CBD or other chemicals contained in marijuana) for the treatment of seizures, anxiety, and other conditions.

In certain embodiments, the in-line drug delivery device of the disclosure may be used to deliver scheduled and controlled substances such as narcotics for the highly controlled dispense of pain medications where dosing is only enabled by doctor or pharmacy communication to the device, and where dosing may only be enabled in a specific location such as the patient's residence as verified by GPS location on the patient's smart phone. This mechanism of highly controlled dispensing of controlled medications can prevent the abuse or overdose of narcotics or other addictive drugs.

Certain benefits of the pulmonary route for delivery of drugs and other medications include a non-invasive, needle-free delivery system that is suitable for delivery of a wide range of substances from small molecules to very large proteins, reduced level of metabolizing enzymes compared to the GI tract and absorbed molecules do not undergo a first pass effect. (A. Tronde, et al., J Pharm Sci, 92 (2003) 1216-1233; A. L. Adjei, et al., Inhalation Delivery of Therapeutic Peptides and Proteins, M. Dekker, New York, 1997). Further, medications that are administered orally or intravenously are diluted through the body, while medications given directly into the lungs may provide concentrations at the target site (the lungs) that are about 100 times higher than the same intravenous dose. This is especially important for treatment of drug resistant bacteria, drug resistant tuberculosis, for example and to address drug resistant bacterial infections that are an increasing problem in the ICU.

Another benefit for giving medication directly into the lungs is that high, toxic levels of medications in the blood stream their associated side effects can be minimized. For example, intravenous administration of tobramycin leads to very high serum levels that are toxic to the kidneys and therefore limits its use, while administration by inhalation significantly improves pulmonary function without severe side effects to kidney functions. (Ramsey et al., Intermittent administration of inhaled tobramycin in patients with cystic fibrosis. N Engl J Med 1999; 340:23-30; MacLusky et al., Long-term effects of inhaled tobramycin in patients with cystic fibrosis colonized with Pseudomonas aeruginosa. Pediatr Pulmonol 1989; 7:42-48; Geller et al., Pharmacokinetics and bioavailability of aerosolized tobramycin in cystic fibrosis. Chest 2002; 122:219-226.)

As described, effective delivery of droplets deep into the lung airways require droplets that are less than about 5-6 microns in diameter, specifically droplets with mass mean aerodynamic diameters (MMAD) that are less than about 5 microns. The mass mean aerodynamic diameter is defined as the diameter at which 50% of the droplets by mass are larger and 50% are smaller. In certain aspects of the disclosure, in order to deposit in the alveolar airways, droplets in this size range must have momentum that is sufficiently high to permit ejection out of the device, but sufficiently low to overcome deposition onto the tongue (soft palate) or pharynx.

In other aspects of the disclosure, methods for generating an ejected stream of droplets for delivery to the pulmonary system of user using the droplet delivery devices of the disclosure are provided. In certain embodiments, the ejected stream of droplets is generated in a controllable and defined droplet size range. By way of example, the droplet size range includes at least about 50%, at least about 60%, at least about 70%, at least about 85%, at least about 90%, between about 50% and about 90%, between about 60% and about 90%, between about 70% and about 90%, etc., of the ejected droplets are in the respirable range of below about 5 μm.

In other embodiments, the ejected stream of droplets may have one or more diameters, such that droplets having multiple diameters are generated so as to target multiple regions in the airways (mouth, tongue, throat, upper airways, lower airways, deep lung, etc.) By way of example, droplet diameters may range from about 1 μm to about 200 μm, about 2 μm to about 100 μm, about 2 μm to about 60 μm, about 2 μm to about 40 μm, about 2 μm to about 20 μm, about 1 μm to about 5 μm, about 1 μm to about 4.7 μm, about 1 μm to about 4 μm, about 10 μm to about 40 μm, about 10 μm to about 20 μm, about 5 μm to about 10 μm, and combinations thereof. In particular embodiments, at least a fraction of the droplets have diameters in the respirable range, while other droplets may have diameters in other sizes so as to target non-respirable locations (e.g., larger than 5 μm). Illustrative ejected droplet streams in this regard might have 50%-70% of droplets in the respirable range (less than about 5 μm), and 30%-50% outside of the respirable range (about 5 μm-about 10 μm, about 5 μm-about 20 μm, etc.)

In another embodiment, methods for delivering safe, suitable, and repeatable dosages of a medicament to the pulmonary system using the droplet delivery devices of the disclosure are provided. The methods deliver an ejected stream of droplets to the desired location within the pulmonary system of the subject, including the deep lungs and alveolar airways.

In certain aspects of the disclosure, an in-line droplet delivery device for delivery an ejected stream of droplets to the pulmonary system of a subject is provided. The in-line droplet delivery device generally includes a housing with an outlet for ejected droplets, a reservoir in fluid communication with the outlet, an ejector mechanism in fluid communication with the reservoir and the outlet, and preferably at least one differential pressure sensor positioned within the housing. The differential pressure sensor is configured to electronically breath activate the ejector mechanism upon sensing a pre-determined pressure change within the housing, and the ejector mechanism is configured to generate a controllable plume of an ejected stream of droplets. The ejected stream of droplets includes, without limitation, solutions, suspensions, or emulsions which have viscosities in a range capable of droplet formation using the ejector mechanism. The ejector mechanism may include a piezoelectric or other electromechanical actuator which is directly or indirectly coupled to an aperture plate having a plurality of openings formed through its thickness. The piezoelectric actuator is operable to oscillate the aperture plate directly or indirectly at a frequency to thereby generate an ejected stream of droplets.

In certain embodiments, the in-line droplet delivery device may include a combination reservoir/ejector mechanism module that may be replaceable or disposable either on a periodic basis, e.g., a daily, weekly, monthly, as-needed, etc. basis, as may be suitable for a prescription or over-the-counter medication. The reservoir may be prefilled and stored in a pharmacy for dispensing to patients or filled at the pharmacy or elsewhere by using a suitable injection means such as a hollow injection syringe driven manually or driven by a micro-pump. The syringe may fill the reservoir by pumping fluid into or out of a rigid container or other collapsible or non-collapsible reservoir. In certain aspects, such disposable/replaceable, combination reservoir/ejector mechanism module may minimize and prevent buildup of surface deposits or surface microbial contamination on the aperture plate, owing to its short in-use time.

The present disclosure also provides an in-line droplet delivery device that is altitude insensitive. In certain implementations, the in-line droplet delivery device is configured to be insensitive to pressure differentials that may occur when the user travels from sea level to sub-sea levels and at high altitudes, e.g., while traveling in an airplane where pressure differentials may be as great as 4 psi. In certain implementations of the disclosure, the in-line droplet delivery device may include a superhydrophobic filter, optionally in combination with a spiral vapor barrier, which provides for free exchange of air into and out of the reservoir, while blocking moisture or fluids from passing into the reservoir, thereby reducing or preventing fluid leakage or deposition on aperture plate surfaces.

While the invention has been described with reference to exemplary embodiments, it will be understood by those skilled in the art that various changes may be made and equivalents may be substituted for elements thereof without departing from the scope of the push mode invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the push mode invention will include all embodiments falling within the scope of the appended claims.

What is claimed:

1. A droplet delivery device comprising:
a housing with an outlet configured for droplets to be ejected from the droplet delivery device;
a reservoir configured to supply a volume of fluid and in fluid communication with the outlet;
an ejector mechanism in fluid communication with the reservoir and the outlet;
an accelerometer coupled to a power source and to the housing;
a microcontroller unit coupled to the accelerometer and programmed to communicate a confirmation of sufficient movement of the droplet delivery device to mix the volume of fluid; and
a feedback unit communicatively coupled to the microcontroller unit providing one or both of display and sound notification in response to receiving the confirmation of sufficient movement.

2. The droplet delivery device of claim 1, wherein the accelerometer is configured to measure at least one of acceleration and orientation on multiple axes.

3. The droplet delivery device of claim 2, wherein the accelerometer is configured to measure at least one of acceleration and orientation on at least three orthogonal axes.

4. The droplet delivery device of claim 3, wherein the ejector mechanism includes an electromechanical actuator.

5. The droplet delivery device of claim 2, wherein the ejector mechanism includes an electromechanical actuator.

6. The droplet delivery device of claim 1, wherein the ejector mechanism includes an electromechanical actuator.

7. The droplet delivery device of claim 1, wherein the reservoir contains a fluid including a drug.

8. The droplet delivery device of claim 1, wherein the reservoir contains a fluid including nicotine or a cannabinoid.

9. The droplet delivery device of claim 1, wherein the reservoir contains a fluid including a therapeutic agent.

10. The droplet delivery device of claim 1, wherein the feedback unit is configured to provide a voice notification.

11. The droplet delivery device of claim 1, wherein the microcontroller unit is programmed to initiate the accelerometer to determine at least one of acceleration and orientation at predetermined intervals.

12. The droplet delivery device of claim 11, wherein the microcontroller unit is programmed to wake the accelerometer to initiate the accelerometer to determine at least one of acceleration and orientation at predetermined intervals.

13. The droplet delivery device of claim 12, wherein the microcontroller is programmed to communicate directions for moving the droplet delivery device to the feedback unit based on one or more determinations of at least one of acceleration and orientation at one or more predetermined intervals.

14. The droplet delivery device of claim 11, wherein the microcontroller is programmed to communicate directions for moving the droplet delivery device to the feedback unit based on one or more determinations of at least one of acceleration and orientation at one or more predetermined intervals.

15. The droplet delivery device of claim 1, further comprising a recordable memory coupled to the microcontroller unit, wherein the microcontroller unit is programmed to store orientation data from the accelerometer relative to time in the recordable memory.

16. The droplet delivery device of claim 1, further comprising a recordable memory coupled to the microcontroller unit, wherein the microcontroller unit is programmed to store movement data from the accelerometer to the recordable memory.

17. The droplet delivery device of claim 1, wherein the ejector mechanism is configured to produce droplets with an average ejected droplet diameter of less than about 6 microns.

18. The droplet delivery device of claim 1, wherein the ejector mechanism is configured to produce droplets with an average ejected droplet diameter of less than about 5 microns.

19. The droplet delivery device of claim 1, wherein the ejector mechanism is configured to produce droplets with an average ejected droplet diameter of more than about 1 micron to less than about 6 microns.

20. The droplet delivery device of claim 1, wherein the ejector mechanism is configured to produce droplets with an average ejected droplet diameter of more than about 1 micron to less than about 5 microns.
